(19) United States
(12) Patent Application Publication
(10) Pub. No.: US 2019/0125410 A1
(43) Pub. Date: May 2, 2019

(54) EXPANDABLE SURGICAL FIXATION ASSEMBLIES AND METHOD OF USE

(71) Applicant: K2M, Inc., Leesburg, VA (US)

(72) Inventor: Brittany Harwell, Warrenton, VA (US)

(21) Appl. No.: 16/095,984

(22) PCT Filed: Apr. 26, 2017
(86) PCT No.: PCT/US2017/029621

§ 371 Date: Oct. 24, 2018

Related U.S. Application Data
(60) Provisional application No. 62/327,542, filed on Apr. 26, 2016.

(51) Int. Cl. A61B 17/70; A61B 17/84; A61B 17/86

(52) U.S. Cl. CPC A61B 17/7035; A61B 2017/8655; A61B 17/86; A61B 17/844

(57) ABSTRACT

A surgical fixation assembly includes a casing and a probe. The casing defines a longitudinal axis and includes a head and a shaft extending from the head. The shaft includes a first arm and a second arm. The probe includes a wedge and a head that are coupled together and supported within the casing, the wedge and the head positioned to move relative to the casing for selectively securing the casing to osseous tissue. The wedge is supported for rotation and translation along the first and second arms of the casing to deflect the first and second arms radially and axially relative to the longitudinal axis of the casing.

### CROSS-REFERENCE TO RELATED APPLICATIONS

The present application is a national stage entry of International Application No. PCT/US2017/029621 filed on Apr. 26, 2017 which claims the benefit of U.S. Provisional Patent Application No. 62/327,542, filed on Apr. 26, 2016, the entire contents of which are incorporated by reference herein.

### TECHNICAL FIELD

The present disclosure relates to surgical fixation assemblies. More particularly, the present disclosure relates to expandable fixation assemblies and methods for attaching the expandable fixation assemblies to osseous tissue (e.g., a vertebral body of a human spine).

### BACKGROUND

The spine is a highly flexible structure capable of a high degree of curvature and twist in nearly every direction. An adult spine generally has twenty-four vertebrae that can be categorized into three major sections: the cervical spine, the thoracic spine, and the lumbar spine. The cervical spine includes the upper seven vertebrae, the thoracic spine includes the next twelve vertebrae, and the lumbar spine includes the final five vertebrae. Below the lumbar spine is a bone called the sacrum, which is part of the pelvis. Muscles and ligaments attach to a slender projection from the back of the vertebrae known as the spinous process. Housed within a narrow channel in the center of the spine is the spinal cord to which nerves of the body's nervous system are connected.

Spinal pathologies, whether the result of genetic or developmental irregularities, trauma, chronic stress, tumors, and/or disease, can limit a range of motion of the spine and/or threaten critical elements of the nervous system.

A variety of systems have been devised to correct such spinal pathologies, and depending on how such systems are coupled to the spine, the systems may be classified as anterior, posterior, or lateral. For example, posterior systems generally include rods that are fixed to adjacent vertebrae with fixation assemblies, such as pedicle screws or hooks, on either side of one or more spinous processes. Achieving optimum alignment between the system and the vertebrae is limited by the range of motion achievable by the system; in other words, the greater the range of motion achievable by the system, the closer the system may be aligned with the vertebrae. Besides the limited range of motion achievable by current systems, the current systems often can be complex and difficult to manipulate.

### SUMMARY

According to one aspect, the present disclosure relates to a surgical fixation assembly including a casing and a probe. The casing defines a longitudinal axis and includes a head and a shaft extending from the head. The shaft includes a first arm and a second arm. The probe includes a wedge and a head that are coupled together and supported within the casing, the wedge and the head positioned to move relative to the casing for selectively securing the casing to osseous tissue. The wedge is supported for rotation and translation along the first and second arms of the casing to deflect the first and second arms radially and axially relative to the longitudinal axis of the casing.

In some embodiments, an outer surface of the casing may include one or more threads configured to facilitate securement of the casing to osseous tissue.

In certain embodiments, the head of the casing may include a threaded inner surface and the head of the probe may include a threaded outer surface that threadably engages the threaded inner surface of the head of the casing to enable the probe to rotate relative to the casing.

According to some embodiments, the head of the casing may have a spherical configuration.

In certain embodiments, rotation of the probe relative to the casing may cause the probe to translate in a proximal direction.

In some embodiments, the first and second arms of the casing may deflect radially outward and proximally as the probe translates in a proximal direction.

According to certain embodiments, the wedge may contact an inner surface of the first and second arms as the wedge translates therealong.

In some embodiments, the shaft of the casing may define a recess therein positioned to receive the wedge of the probe. The recess may have a frustoconical configuration.

In embodiments, the shaft of the casing may define a transverse bore therethrough that separates the first and second arms of the shaft.

According to another aspect of the present disclosure, a surgical fixation system includes a rod-connecting housing, a casing, and a probe. The casing defines a longitudinal axis and includes a head and a shaft that extends from the head. The head supports the rod-connecting housing. The shaft includes a first arm and a second arm. The probe includes a wedge and a head that are coupled together and supported within the casing. The wedge and the head positioned to move relative to the casing for selectively securing the casing to osseous tissue. The wedge is supported for rotation and translation along the first and second arms of the casing to deflect the first and second arms radially and axially relative to the longitudinal axis of the casing.

In some embodiments, the head of the casing may have a spherical configuration to enable the rod-connecting housing to polyaxially pivot about the head of the casing.

In certain embodiments, the wedge and the head may be coupled together by a shaft of the probe. The shaft of the probe may include separate portions that coupled together within the casing to secure the wedge and the head together within the casing.

In some embodiments, a surgical fixation kit may include a plurality of one or more of the wedge, the head, and/or the shaft. Two of the plurality may have different lengths such that the probe can be provided with different lengths while supported within the casing.

According to still another aspect of the present disclosure, a method of inserting a surgical fixation assembly into osseous tissue is provided. The method includes inserting a probe and a casing of the surgical fixation assembly into the osseous tissue to a predetermined depth within the osseous tissue while the probe is positioned within the casing. The method further includes rotating the casing relative to the probe to secure threads of the casing to the osseous tissue when the surgical fixation assembly is inserted to the predetermined depth, rotating the probe relative to the casing to rotate and translate a wedge of the probe relative to the casing, and engaging the wedge with first and second arms of the casing to deflect the first and second arms in a radial and axial direction to secure the first and second arms within osseous tissue.

### BRIEF DESCRIPTION OF THE DRAWINGS

The accompanying drawings, which are incorporated in and constitute a part of this specification, illustrate one or more embodiments of the disclosure and, together with a general description of the disclosure given above, and the detailed description given below, serve to explain the principles of the disclosure, wherein:

FIG. 1A is a perspective view of a fixation assembly with a casing and a probe of the fixation member shown in a first position according to an embodiment of the present disclosure;

FIG. 1B is a perspective view of the fixation assembly of FIG. 1A with the casing and the probe thereof shown in a second position;

FIG. 2 is a perspective view of the casing of the fixation assembly of FIGS. 1A and 1B with the casing shown in the first position thereof;

FIG. 3 is a perspective view of the probe of the fixation assembly of FIGS. 1A and 1B;

FIG. 4 is a longitudinal, cross-sectional view taken along section line 4-4 shown in FIG. 1A;

FIG. 5A is a cross-sectional view taken along section line 5A-5A shown in FIG. 1A;

FIG. 5B is a cross-sectional view taken along section line 5B-5B shown in FIG. 1B;

FIG. 6 is a perspective view of a portion of a fixation system according to another embodiment of the present disclosure;

FIG. 7 is a perspective view of a portion of a fixation system according to yet another embodiment of the present disclosure; and

FIG. 8 is a perspective view of one example of a spinal plate for use with the fixation assembly of FIGS. 1A and 1B.

### DETAILED DESCRIPTION

In general, the present disclosure relates to expandable fixation assemblies for securing surgical systems to anatomical features of a body. The fixation assemblies are secured to osseous tissue, for example, a pedicle of a vertebral body, iliac of the pelvis, or the like. The expandable fixation assemblies are configured to reduce insertion time and effort required for securing to osseous tissue.

Embodiments of the presently disclosed expandable fixation assemblies are described in detail with reference to the drawings, in which like reference numerals designate identical or corresponding elements in each of the several views. Well known functions or constructions are not described in detail so as to avoid obscuring the present disclosure in unnecessary detail.

As used herein, the term “clinician” refers to a doctor, nurse, or other care provider and may include support personnel. The term “distal” refers to structure that is farther from a clinician, while the term “proximal” refers to structure that is closer to the clinician. Further, directional terms such as front, rear, upper, lower, top, bottom, distal, proximal, and similar terms are used to assist in understanding the description and are not intended to limit the present disclosure.

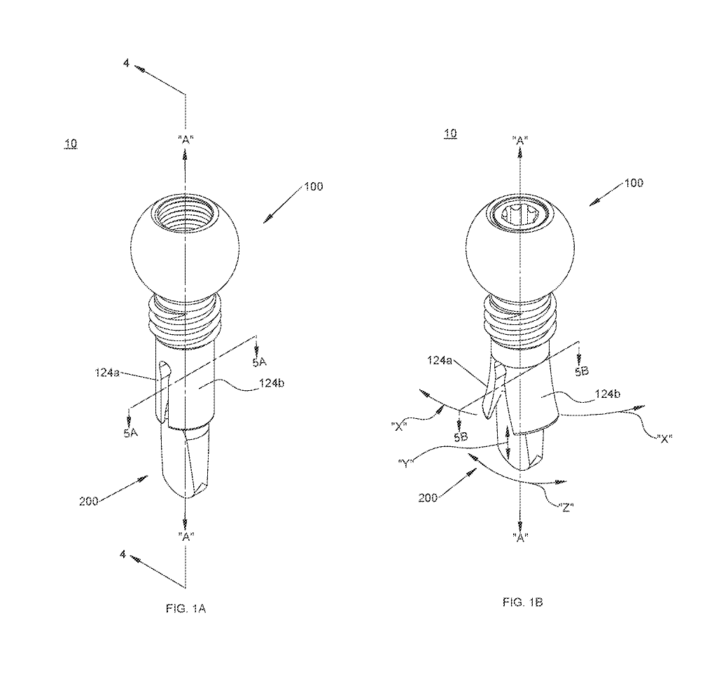

Referring initially to FIGS. 1A and 1B, a fixation assembly, generally designated 10, includes a casing 100 and a probe 200. The casing 100 defines a longitudinal axis “A” and is configured to rotatably receive the probe 200, as indicated by arrows “Z,” to enable the probe 200 to translate along the longitudinal axis “A” and relative to the casing 100, as indicated by arrows “Y.” As the probe 200 moves in relation to the casing 100, the probe 200 causes the casing 100 (and thus the fixation assembly 10) to move between a first position (FIG. 1A) and a second position (FIG. 1B) for selectively securing the fixation assembly 10 in osseous tissue. More particularly, while the casing 100 is in the first position, the fixation assembly 10 is configured to be inserted into and removed from the osseous tissue, and while in the second position, the casing 100 is configured to secure the fixation assembly 10 to osseous tissue.

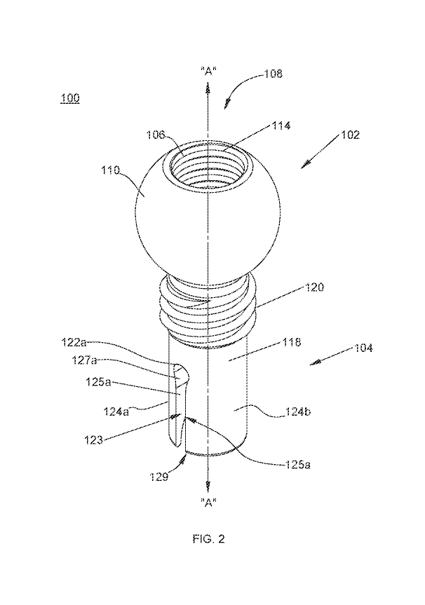

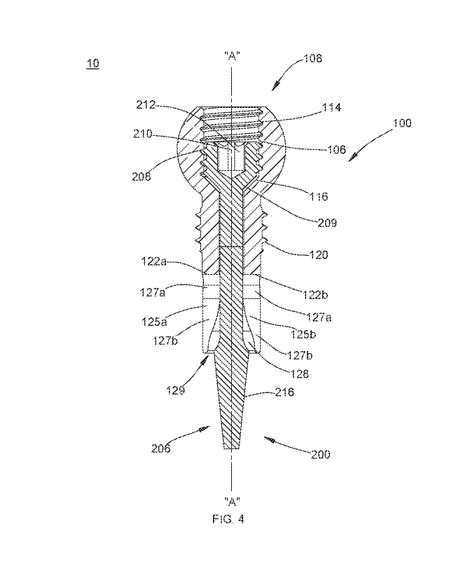

Referring to FIGS. 2 and 4, the casing 100 of the fixation assembly 10 generally includes a head 102 at a proximal portion of the casing 100 and a shaft 104 that extends distally from a distal portion of the head 102 to a distal portion of the casing 100. The head and shaft 102, 104 of the casing 100 collectively define a passageway 108 through the casing 100 for rotatably and translatably receiving the probe 200.

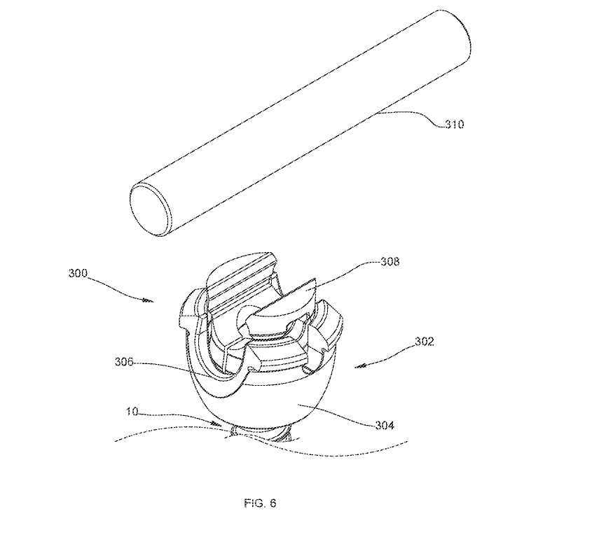

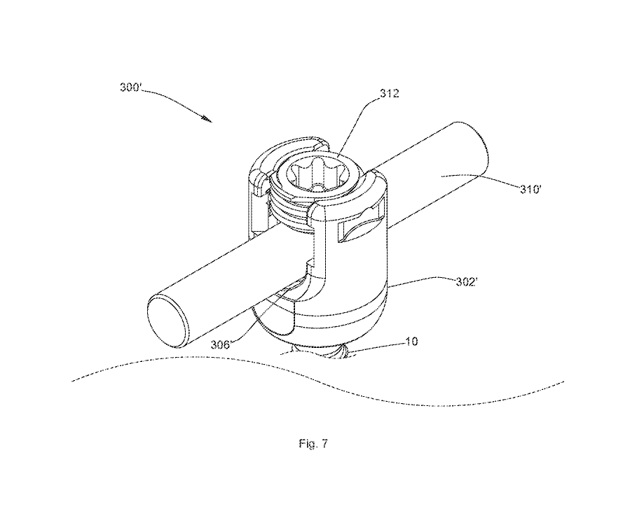

The head 102 of the casing 100 has an inner surface 106 and an outer surface 110. The outer surface 110 of the head 102 has a spherical configuration to facilitate multi or polyaxial movement of a rod-connecting housing, such as a taper-lock type housing assembly 302 (see FIG. 6) or a set-screw type housing assembly 302 (see FIG. 7), relative to the fixation assembly 10; though the outer surface 110 may have any suitable configuration such as, without limitation, polygonal or elliptical configurations. The inner surface 106 of the head 102 includes threads 114 that extend along the passageway 108 from a tapered surface 116 (FIG. 4) at a distal portion of the head 102 to a proximal portion of the head 102. Although the tapered surface 116 is seen in FIG. 4 as having a conical configuration, the tapered surface 116 may include any suitable configuration.

With reference to FIGS. 2 and 4, the shaft 104 of the casing 100 includes an outer surface 118 having threads 120 disposed about a proximal portion of the shaft 104. The outer surface 118 or 110 of the casing may include surface texturing. The threads 120 of the shaft 104, which may be or include ribs or ridges, extend distally along the outer surface 118 of the shaft 104 (e.g., helically and/or a longitudinally spaced locations) and function to engage osseous tissue for securing the casing 100 to the osseous tissue. The shaft 104 includes a first apex 122a and a second apex 122b that are disposed at a location distal to the threads 120 and in mirrored relation about the longitudinal axis “A” of the casing 100. The shaft 104 further includes first and second arms 124a, 124b that extend from the first and second apices 122a, 122b, respectively, and which are spaced-apart by a transverse opening 123 that extends through the distal portion of the casing 100. Although seen extending along a partial length of the shaft 104, the first and/or second arms 124a, 124b may extend along an entire length of the shaft 104 and/or along different lengths relative to one another.

With continued reference to FIGS. 2 and 4, each of the first and second arms 124a, 124b of the shaft 104 extends distally along curvilinear side surfaces 125a, 125b thereof, respectively, toward a distal portion of the shaft 104. More particularly, the first apex 122a of the shaft 104 connects the curvilinear side surface 125a of the first arm 124a to the curvilinear side surface 125a of the second arm 124b. The curvilinear side surfaces 125a of the first and second arms 124a, 124b are disposed in mirrored relation about the transverse opening 123 of the shaft 104. Similarly, the second apex 122b of the shaft 104 connects the curvilinear side surface 125b of the first arm 124a to the curvilinear side surface 125b of the second arm 124b. The curvilinear side surfaces 125b of the first and second arms 124a, 124b are also disposed in mirrored relation about the transverse opening 123 of the shaft 104. Each curvilinear side surface 125a, 125b of the respective first and second arms 124a, 124b includes an arcuate portion 127a and a planar portion 127b that extends distally from the arcuate portion 127a.

With reference to FIGS. 1B, 2 and 4, the first and second apices 122a, 122b of the shaft 104, the curvilinear side surfaces 125a, 125b of the first and second arms 124a, 124b of the shaft 104, and/or the transverse opening 123 of the shaft 104 function to enable the first and second arms 124a, 124b to deflect relative to the longitudinal axis “A” of the casing 100. More particularly, the first and second arms 124a, 124b are configured to deflect radially and axially relative to the longitudinal axis “A” of the casing 100, as indicated by arrows “X” seen in FIG. 1B.

With reference again to FIG. 4, each of the first and second arms 124a, 124b of the shaft 104 of the casing 100 include a curved inner surface 128 that may be smooth. The curved inner surfaces 128 of the first and second arms 124a, 124b collectively define an arcuate recess 129 in a distal portion of the shaft 104 for selectively engaging the probe 200 as the probe 200 moves (e.g., rotates and/or translates) relative to the casing 100. The arcuate recess 129 may be frustoconical or substantially frustoconical.

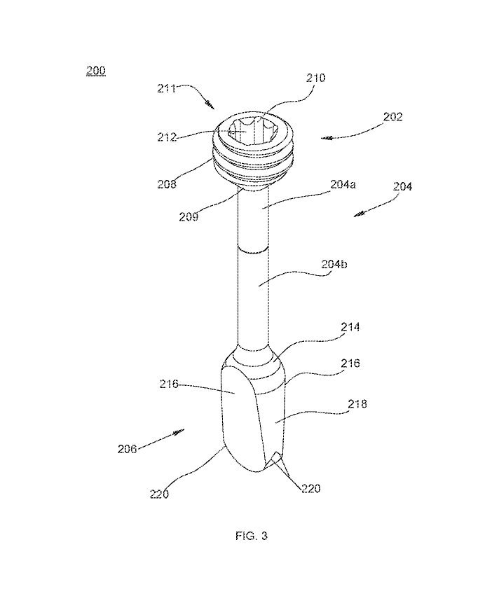

Referring now to FIG. 3, the probe 200 of the fixation assembly 10 generally includes a head 202 at a proximal portion of the probe 200 for movably supporting the probe 200 in the casing 100, a wedge 206 at a distal portion of the probe 200 for facilitating insertion of the fixation assembly 10 into osseous tissue and selectively deflecting the first and second arms 124a, 124b of the casing 100, and a shaft 204 that extends between the head 202 and the wedge 206.

With reference to FIGS. 2-4, the head 202 of the probe 200 includes an engagement surface 210 that defines a driving recess 211 in a proximal portion of the head 202. Although shown as having a hexolobular configuration, the driving recess 211 may have any suitable configuration with any suitable cross-sectional shape (e.g., triangular, rectangular, pentagonal, etc.) The engagement surface 210 includes projections 212 disposed along a periphery of the engagement surface 210 to enable engagement with a screw driver (not shown), for driving the probe 200 relative to the casing 100. The head 202 includes threads 208 disposed along an outer surface of the head 202 for threadably engaging the threads 114 of the inner surface 106 of the head 102 of the casing 100. The threads 208 of the head 202 and of the probe 200 extend distally from a proximal portion of the head 202 toward a tapered surface 209 located along a distal portion of the head 202 that is configured to engage the tapered surface 116 of the inner surface 106 of the head 102 of the casing 100.

The shaft 204 of the probe 200 comprises a first member 204a and a second member 204b that are coupled together within the casing 100. The first member 204a of the shaft 204 has a proximal portion that extends distally from a distal portion of the head 202 of the probe 200. The first member 204a also has a distal portion that is configured to be inserted through a proximal portion of the casing 100. The second member 204b extends proximally from a proximal portion of the wedge 206 to a proximal portion of the second member 204b. The proximal portion of the second member 204b is configured to be inserted through a distal portion of the casing 100.

Briefly, to insert the probe 200 into the casing 100, the distal portion of the first member 204a of the probe 200 is advanced distally through the proximal portion of the casing 100 and the proximal portion of the second member 204b is advanced proximally through the distal portion of the casing 100 (e.g., at opposite ends of the casing 100). The first and second members 204a, 204b are then engaged with one another in the passageway 108 of the casing 100 and coupled using any known securement technique such as threading, fastening, adhesion, snap-fit, friction-fit, interference-fit, etc., to couple the head 202 and wedge 206 of the probe 200 together in the passageway 108 of the casing 100. In certain embodiments, the first and second members 204a, 204b may be integrally or monolithically formed.

Referring to FIG. 3, the wedge 206 of the probe 200 includes a neck 214 located along a proximal portion of the wedge 206. The neck 214 of the wedge 206 connects a pair of tapered surfaces 216 and a pair of curved surfaces 218 that extend distally from the neck 214, and which alternate about an outer surface of the wedge 206. The wedge 206 further includes a pair of cut edges 220 that extend from the curved surfaces 218, respectively, and are disposed between the pair of tapered surfaces 216 at a distal end of the wedge 206. The distal end of the wedge 206 may be blunt. In some embodiments, the distal end of the wedge 206 is pointed to penetrate tissue.

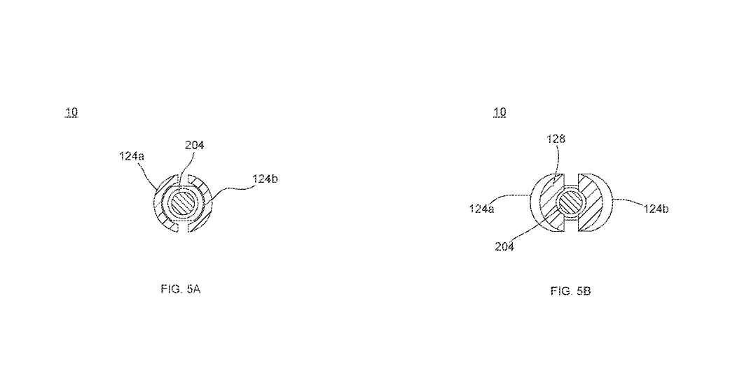

In use, with reference to FIGS. 1A-5B, a hole can be drilled or otherwise formed in osseous tissue using known devices and techniques (e.g., punching, cutting, coring, etc.). While in the first position (FIG. 1A), the fixation assembly 10 is configured to be inserted into the hole and advanced therein.

Upon achieving sufficient insertion depth, the casing 100 can be rotated relative to the probe 200 (e.g., the probe 200 may be rotationally fixed by a rotational counterforce applied by a screw driver engaged with the probe 200—not shown) to enable the threads 120 on the outer surface of the casing 100 to threadably engage the osseous tissue to fix the casing 100 to the osseous tissue. Alternatively, the casing 100 may rotated with the probe 200 (e.g., the entire fixation assembly 10) to secure the threads 120 of the casing 100 to the osseous tissue. The head 102 of the casing 100 may be configured to contact osseous tissue to define an insertion depth limit. More particularly, the head 102 may have a cross-section which is wider than a cross-section defined by the shaft 104 of the casing 100 which, as the fixation assembly 10 is inserted into osseous tissue, contacts the osseous tissue. As the head 102 contacts the osseous tissue, the head 102 may function as a guide to ensure proper placement of the fixation assembly 10 by limiting the insertion depth of the fixation assembly 10.

With the casing 100 secured in the osseous tissue via the threads 120 of the casing 100, the probe 200 of the fixation assembly 10 can be rotated relative to the casing 100 by a driving tool, such as a screw driver, engaged with the driving recess 211 of the probe 200 to cause the probe 200 to move proximally along the longitudinal axis “A” relative to the casing 100. As the probe 200 moves proximally along the longitudinal axis “A”, the wedge 206 of the probe 200 (e.g., the neck 214 and/or the curved surfaces 218 thereof) engages the curved inner surfaces 128 of the arms 124a, 124b of the casing 100, to cause the arms 124a, 124b of the casing 100 to deflect (e.g., simultaneously) radially outward and upward (e.g., proximally) to secure the arms 124a, 124b of the casing 100 to the surrounding osseous tissue to facilitate securement of the fixation assembly 10 to the osseous tissue.

With the first and second arms 124a, 124b of the casing 100 in an at least partially deflected position (FIG. 1B), an amount of deflection of the first and second arms 124a, 124b can be adjusted as desired and/or even reset to an undeflected position (FIG. 1A) via rotation of the probe 200 relative to the casing 100 about the longitudinal axis “A” (e.g., in clockwise and/or counterclockwise directions) of the casing 100. In particular, the probe 200 can be rotated relative to the casing 100 by a driving tool, such as a screw driver, to cause the probe 200 to move distally (and/or proximally) along the longitudinal axis “A” relative to the casing 100. As the probe 200 moves distally along the longitudinal axis “A,” the wedge 206 of the probe 200 disengages from the curved inner surfaces 128 of the arms 124a, 124b of the casing 100 to cause the arms 124a, 124b of the casing 100 to retract radially inward and downward (e.g., distally). The first and second arms 124a, 124b are configured to bias toward the undeflected position (FIG. 1A). If the first and second arms 124a, 124b are disposed in an undeflected position, the casing 200 may be rotated about the longitudinal axis “A” thereof to disengage the threads 120 of the shaft 104 thereof for removing the fixation assembly 10 from the hole in the osseous tissue.

Advantageously, securement of the fixation assembly 10 is achieved with reduced driving effort as compared to the multiple rotations required to distally advance and secure a traditional bone screw in osseous tissue. Additionally, the fixation assembly 10 may be shorter in length than a traditional bone screw and further configured not to extend into predetermined portions of osseous tissue. For example, if the osseous tissue is a pedicle, then the fixation assembly 10 can have a length that would not extend into the vertebral body interspace.

Referring now to FIG. 6, a fixation system 300 may include the fixation assembly 10 and a housing assembly 302 coupled to the head 102 (see FIG. 2) of the casing 100 for selectively securing a spinal rod 310 to the osseous tissue. More particularly, the housing assembly 302 is coupled to the head 102 of the casing 100 such that the housing assembly 302 is polyaxially movable about the head 102 of the casing 100. The taper lock arrangement of the housing assembly 302 generally includes an outer housing 304 that is slidably movable about an inner collet 308 to selectively secure the spinal rod 310 within a U-shaped saddle 306 defined therein. Although the housing assembly 302 is shown as a taper lock arrangement in FIG. 6, the housing assembly 302 can have any suitable configuration such as a set screw type arrangement, as shown in FIG. 7.

Briefly, as seen in FIG. 7, a fixation system 300′generally includes a housing assembly 302′in which a set screw 312 is threadably received to secure a spinal rod 310 within a U-shaped saddle 306′defined therein. The housing assembly 302′can be, for example, configured to mount onto the fixation assembly 10.

For a more detailed description of example taper lock and/or set screw type housing assemblies, reference can be made to U.S. Pat. Nos. 9,393,049 and 8,814,919, the entire disclosures of each of which are incorporated by reference herein.

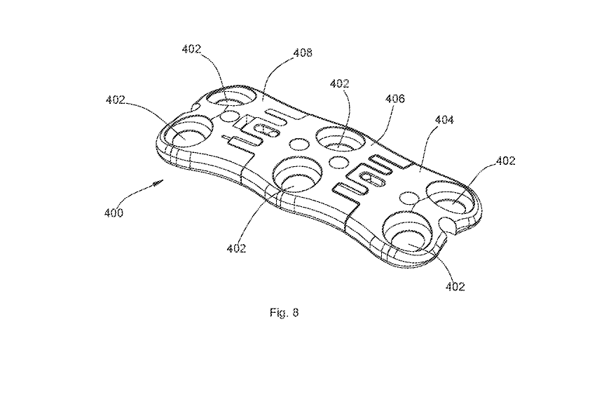

Referring now to FIG. 8, the presently disclosed fixation assemblies can, in some embodiments, be included with any suitable spinal plate, for example to secure the spinal plate across one or more vertebrae. As illustrated, a spinal plate 400 generally defines one or more apertures or openings 402 therethrough that receive fixation assemblies, such as bone screws, for securing the spinal plate 400 to vertebrae. The presently disclosed fixation assembly 10 may be utilized with, or in place of, such bone screws. Spinal plates, such as spinal plate 400, may have two or more sections that are movable relative to one another. For example, spinal plate 400 includes a first end section 404, a middle section 406, and a second end section 408. For a more detailed description of an example spinal plate, reference can be made to commonly owned U.S. Pat. No. 8,636,738, the entire disclosure of which is incorporated by reference herein.

Any of the presently disclosed embodiments, or components thereof, can be formed of any suitable material or combinations of materials such as mixed metallic materials including titanium, titanium alloy, stainless steel, nickel titanium, polyetheretherketone (PEEK), cobalt-chromium, and other known biocompatible materials. Further, the presently disclosed embodiments, or components thereof, can be formed using any suitable technique such as welding, fastening, machining, molding, three-dimensional (3D) printing, etc. Any of the components may be secured together using any known technique such as press-fit, fastening, adhesion, etc.

In embodiments, the outer surface 118 of the shaft 104 of the casing 100 may be textured to engage osseous tissue as the fixation assembly 10 is inserted into the osseous tissue. For example, the outer surface 118 may be configured to have a coarse or rough surface to increase friction between the arms 124a, 124b and the osseous tissue while the arms 124a, 124b are in contact with the osseous tissue.

In some embodiments, the inner surface 106 of the casing 100 and the head 202 of the probe 200 may be configured to engage one or more tools (not shown) simultaneously. More particularly, the head 102 of the casing 100 may be rotatably engaged by a first portion of a tool (e.g., a screw driver—not shown) and the head 202 of the probe 200 may be engaged by a second portion of the tool to maintain the position of the probe 200 relative to the casing 100 as the fixation assembly 10 is inserted into osseous tissue. When the fixation assembly 10 is advanced distally to a desired depth in the osseous tissue, the tool may rotatably engage the probe 200 to move the fixation assembly into the second position to secure the fixation assembly 10 in the osseous tissue.

In some embodiments, the probe 200, the casing 100, and/or components thereof, (e.g., the shaft 204 of the probe 200, the wedge 206 of the probe 200, etc.) may be provided in a kit with multiple components having different configurations (e.g., lengths, shapes, widths, etc.) to accommodate different anatomical structures. For example, the kit may include multiple heads 202 with different shaft portions 204a and/or multiple wedges 206 with different shaft portions 204b (e.g., each of different lengths) so as to be selected and interconnected within the casing 100 to provide a clinician with options for different probe 200 lengths.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. A surgical fixation assembly comprising:
a casing defining a longitudinal axis and including a head and shaft extending from the head, the shaft including a first arm and a second arm; and
a probe including a wedge and a head that are coupled together and supported within the casing, the wedge and the head positioned to move relative to the casing for selectively securing the casing to osseous tissue, the wedge supported for rotation and translation along the first and second arms of the casing to deflect the first and second arms radially and axially relative to the longitudinal axis of the casing.

2. The surgical fixation assembly of claim 1, wherein an outer surface of the casing includes threads configured to facilitate securement of the casing to osseous tissue.

3. The surgical fixation assembly of claim 1, wherein the head of the casing includes a threaded inner surface and the head of the probe includes a threaded outer surface that threadably engages the threaded inner surface of the head of the casing to enable the probe to rotate relative to the casing.

4. The surgical fixation assembly of claim 1, wherein the head of the casing has a spherical configuration.

5. The surgical fixation assembly of claim 1, wherein rotation of the probe relative to the casing causes the probe to translate in a proximal direction.

6. The surgical fixation assembly of claim 5, wherein the first and second arms of the casing deflect radially outward and proximally as the probe translates in a proximal direction.

7. The surgical fixation assembly of claim 1, wherein the wedge contacts an inner surface of the first and second arms as the wedge translates therealong.

8. The surgical fixation assembly of claim 1, wherein the shaft of the casing defines a recess therein positioned to receive the wedge of the probe.

9. The surgical fixation assembly of claim 8, wherein the recess has a frustoconical configuration.

10. The surgical fixation assembly of claim 1, wherein the shaft of the casing defines a transverse bore therethrough that separates the first and second arms of the shaft.

11. A surgical fixation system comprising:
a rod-connecting housing;
a casing defining a longitudinal axis and including a head and shaft extending from the head, the head supporting the rod-connecting housing, the shaft including a first arm and a second arm; and
a probe including a wedge and a head that are coupled together and supported within the casing, the wedge and the head positioned to move relative to the casing for selectively securing the casing to osseous tissue, the wedge supported for rotation and translation along the first and second arms of the casing to deflect the first and second arms radially and axially relative to the longitudinal axis of the casing.

12. The surgical fixation system of claim 11, wherein an outer surface of the casing includes threads configured to facilitate securement with osseous tissue.

13. The surgical fixation system of claim 11, wherein the head of the casing includes a threaded inner surface and the head of the probe includes a threaded outer surface that threadably engages the threaded inner surface of the head of the casing to enable the probe to rotate relative to the casing.

14. The surgical fixation system of claim 11, wherein the head of the casing has a spherical configuration to enable the rod-connecting housing to polyaxially pivot about the head of the casing.

15. The surgical fixation system of claim 11, wherein rotation of the probe relative to the casing causes the probe to translate in a proximal direction.

16. The surgical fixation system of claim 15, wherein the first and second arms of the casing deflect radially outward and proximally as the probe translates in a proximal direction.

17. The surgical fixation system of claim 11, wherein the wedge contacts an inner surface of the first and second arms as the wedge translates therealong.

18. The surgical fixation system of claim 11, wherein the shaft of the casing defines a recess therein positioned to receive the wedge of the probe.

19. The surgical fixation system of claim 11, wherein the shaft of the casing defines a transverse bore therethrough that separates the first and second arms of the shaft.

20. The surgical fixation system of claim 11, wherein the wedge and the head are coupled together by a shaft of the probe.

21. The surgical fixation system of claim 20, wherein the shaft of the probe includes separate portions that couple together within the casing to secure the wedge and the head together within the casing.

22. A surgical fixation kit including the surgical fixation system of claim 21, wherein the kit includes a plurality of at least one of the wedge, the head, or the shaft, and wherein at least two of the plurality have different lengths such that the probe can be provided with different lengths while supported within the casing.

23. A method of inserting a surgical fixation assembly into osseous tissue, the method comprising:
inserting a probe and a casing of the surgical fixation assembly into osseous tissue to a predetermined depth within the osseous tissue while the probe is positioned within the casing;
rotating the casing relative to the probe to secure the threads of the casing to the osseous tissue when the surgical fixation assembly is inserted to the predetermined depth;
rotating the probe relative to the casing to rotate and translate a wedge of the probe relative to the casing; and
engaging the wedge with first and second arms of the casing to deflect the first and second arms in a radial and axial direction to secure the first and second arms within the osseous tissue.
